(19) United States
(12) Patent Application Publication
(10) Pub. No.: US 2013/0137911 A1
(43) Pub. Date: May 30, 2013

(54) CATALYTIC COMPOSITION AND PROCESS FOR THE SELECTIVE DIMERIZATION OF ISOBUTENE

(71) Applicant: IFP ENERGIES NOUVELLES, Rueil-Malmaison Cedex (FR)

(72) Inventors: Lionel MAGNA, Lyon (FR); Helene OLIVIER-BOURBIGOU, Saint Genis-Laval (FR)

(73) Assignee: IFP ENERGIES NOUVELLES, Rueil-Malmaison Cedex (FR)

(21) Appl. No.: 13/688,591

(22) Filed: Nov. 29, 2012

(30) Foreign Application Priority Data
Nov. 30, 2011 (FR) 11/03.654

(51) Int. Cl. B01J 31/02; C07C 2/14

(52) U.S. Cl. CPC B01J 31/0291; C07C 2/14
USPC 585/510; 502/150; 502/167; 502/164

(57) ABSTRACT

Catalytic composition comprising at least one Brønsted acid, designated HB, dissolved in a non-aqueous liquid medium of general formula Q₁⁺A₁⁻, in which Q₁⁺ represents an organic cation and A₁⁻ represents an anion, said composition also comprising an additive Q₂⁺A₂⁻ in which Q₂⁺ represents an organic cation containing at least one alcohol function and A₂⁻ represents an anion. The invention also relates to an isobutene dimerization process using the catalytic composition.

The present invention relates to the dimerization of isobutene. More particularly a subject of the present invention is a novel catalytic composition and a process for the dimerization of isobutene (pure or in a mixture with other hydrocarbons) using this novel catalytic composition.

It is known that the dimers of isobutene (2,4,4-trimethyl-1-(and -2-) pentene) are useful intermediates for the production of different products of commercial interest. By way of example, the higher alcohols, aldehydes and the acids can be mentioned.

2,2,4-Trimethylpentane can be obtained by hydrogenation of trimethylpentenes and constitutes a sought additive for the reformulation of gasolines (absence of sulphur, aromatics and olefins, and low volatility added to a high octane number: Motor octane number (MON) (sic)=Research octane number (RON)=100).

Thus, the selective dimerization of isobutene, followed by hydrogenation of the obtained products to 2,2,4-trimethylpentane having a high octane number, constitutes a useful route which allows
i) the replacement of MTBE (Methyl-Tert-Butyl-Ether: RON=118; MON=100), which is banned for environmental reasons, and
ii) the use of isobutene, originating from the C4 cuts from catalytic cracking or steam cracking processes, a raw material in the production of MTBE.

The dimerization (and also the oligomerization) of isobutene is an exothermic reaction catalyzed by acids. Different acids have been described in the literature such as sulphuric acid, or derivatives thereof, the chlorinated or fluorinated aluminas, zeolites, silica-aluminas etc. However, those most typically used in industry are phosphoric acid, generally supported, and ion-exchange resins (SP-Isoether process licensed by Snamprogetti or UOP's InAlk process).

The main difficulty associated with these processes is obtaining good dimer selectivity. In fact, the exothermicity of the reaction is often difficult to control and leads to the formation of oligomers (essentially C12 olefins and C16 olefins) obtained by parallel reactions starting from isobutene. These oligomers have boiling points which are too high, and are outside, or at the limit, of the specifications required for reformulated gasolines. Moreover, these oligomers contribute to deactivation of the catalysts.

Different works in the literature describe certain solutions for minimizing the formation of these oligomers.

In the case of the ion-exchange resins (Amberlyst-15 or -35 type), the use of a diluent (or solvent) is often recommended. The dimer selectivity depends on the choice of this solvent. The most efficient additives are the alcohols (U.S. Pat. No. 5,877,372; U.S. Pat. No. 4,100,220), which lead to the co-production of ethers, or ethers (in U.S. Pat. No. 4,447,668, MTBE, ETBE, etc.). Mention may be made of the works by Snamprogetti (M. Marchionna et al. Catal. Today, 65 (2001) 397-403, GB 2 325 237) studying the influence of the addition of MTBE or MeOH with the objective of reusing the existing units of MTBE. Useful trimethylpentene selectivities can thus be achieved but often with less than 85% isobutene conversion.

International patent application WO-A-01/51 435 describes a series of processes in which isobutene is produced by dehydration of tert-butyl alcohol. The isobutene is preferentially dimerized by an Amberlyst A-15® type resin in the presence of tert-butyl alcohol (selectivity promoter) and alkane (butane or isobutane) as diluent. The presence of a hindered alcohol discourages the formation of ether but also reduces the rate of reaction.

All the processes described previously have limitations such as the risks of premature deactivation of the catalyst by “clogging” with the heavier oligomers or also the need to use organic additives to control the selectivity of the reaction, organic additives which are often converted and consumed during the reaction and are therefore not recyclable.

The non-aqueous ionic liquids of composition Q⁺A⁻ have been the subject of several articles (for example, H. Olivier-Bourbigou et al., Appl. Catal. A: General, 2010, 1-56). They find numerous applications as solvents for catalysis by transition metals or as extraction solvents for carrying out liquid-liquid extractions.

The patent application WO-A-00/16902 describes the use of an ionic liquid free of Lewis acidity obtained by reaction of a nitrogen-containing (for example an amine or a quaternary ammonium) or phosphorus-containing compound with a Brønsted acid in quantities such that the ratio of said nitrogen-containing or phosphorus-containing compound to the acid is less than 1. These media are used to catalyze in particular the alkylation of benzene with 1-decene.

It has also been described in the application FR2829039 that the addition of at least one Brønsted acid, designated HB, in a non-aqueous liquid medium (“molten salt” type medium) comprising at least one organic cation Q⁺ and an anion A⁻ and in which, when A and B are identical, the molar ratio of the Brønsted acid to the ionic liquid is less than 1/1, leads to liquid compositions which can be used as catalysts and solvents for acid catalysis reactions.

In this patent application, it was mentioned that the catalytic composition described could be used more particularly in the alkylation of aromatic hydrocarbons, but also in the oligomerization of olefins, the dimerization of isobutene, the alkylation of isobutane by olefins, the isomerization of n-paraffins to iso-paraffins and the isomerization of n-olefins to iso-olefins.

The use of the ionic liquids as solvents and acid catalysts for the selective dimerization of isobutene has also been described in the U.S. Pat. No. 7,256,152. The advantage of these liquid catalytic systems for the dimerization reaction of isobutene to isooctenes is that they are not very miscible with the reaction products which can therefore be separated by decantation. The catalytic phase can then be recycled and reused, the consumption of catalyst is thus reduced. However, these systems also have limitations. Like heterogeneous catalysts, it is sometimes difficult to maintain good selectivity of dimerization products at a high isobutene conversion.

A subject of the present invention is to provide a novel catalytic composition making it possible to improve the selectivity of the isobutene dimerization reaction.

Furthermore, the catalytic composition can be recycled without a significant lowering of its catalytic activity and without a significant lowering of its dimer selectivity. The additive included in the catalytic composition according to the invention is therefore neither converted nor consumed during the reaction and is therefore recyclable. It is therefore not necessary to continuously inject an additive.

More particularly, the present invention relates to a catalytic composition comprising at least one Brønsted acid, designated HB, dissolved in a non-aqueous liquid medium of general formula Q₁⁺A₁⁻, in which Q₁⁺ represents an organic cation and A₁⁻ represents an anion, said composition also comprising an additive Q₂⁺A₂⁻ in which Q₂⁺ represents an organic cation comprising at least one alcohol function and A₂⁻ represents an anion.

The ionic liquid Q₁⁺A₁⁻, in which the Brønsted acid HB is dissolved, is defined such that Q₁⁺ represents a quaternary ammonium and/or a quaternary phosphonium and/or a trialkylsulphonium and A₁⁻ represents any anion known to be non-coordinating and capable of forming a liquid salt at low temperature, i.e. below 150° C. The additive Q₂⁺A₂⁻ is defined such that Q₂⁺ represents a quaternary ammonium and/or a quaternary phosphonium and/or a trialkylsulphonium comprising at least one alcohol function and A₂⁻ represents any anion known to be non-coordinating and capable of forming a liquid salt at low temperature, i.e. below 150° C.

The anions A₁⁻ or A₂⁻ considered in the invention are preferably chosen from the following anions: tetrafluoroborate, tetraalkylborates, hexafluorophosphate, hexafluoroantimonate, alkylsulphonates and arylsulphonates (for example methylsulphonate or tosylate), perfluoroalkylsulphonates (for example trifluoromethylsulphonate), fluorosulphonate, sulphates, phosphates, perfluoroacetates (for example trifluoroacetate), perfluoroalkylsulphonamides (for example bis-trifluoromethanesulphonyl amide (N(CF₃SO₂)₂⁻), fluorosulphonamides, perfluoroalkylsulphomethides (for example tris-trifluoromethanesulphonyl methylide (C(CF₃SO₂)₃⁻) and carboranes, A₁ and A₂⁻ being identical or different. Preferably, A₁ and A₂⁻ are identical.

The cations Q₁⁺ and Q₂⁺ considered in the invention are preferably chosen from the quaternary ammoniums and/or the quaternary phosphoniums and/or the trialkylsulphoniums. The chemical nature of Q₁⁺ and Q₂⁺can be identical or different, it is preferably identical. By “identical chemical nature” is meant that if, for example, the cation Q₁⁺ is a quaternary ammonium, Q₂⁺ is also a quaternary ammonium. By “different chemical nature” is meant that if, for example, the cation Q₁⁺ is a quaternary ammonium, Q₂⁺ is chosen from a quaternary phosphonium or a trialkylsulphonium. In any case, the organic cation Q₂⁺ of the additive Q₂⁺A₂⁻ contains at least one alcohol function, while the substituents of the organic cation Q₁⁺ do not have an alcohol function. By “alcohol function” is meant an OH group grafted onto the cation Q₂⁺, either directly onto a carbon of the cation Q₂⁺, or via an aryl, aralkyl, alkyl or cycloalkyl group, preferably comprising from 1 to 12 carbon atoms.

The quaternary ammoniums and/or phosphoniums of general formula Q₁⁺ and Q₂⁺ preferably correspond to the formulae NR¹R²R³R⁴⁺ and PR¹R²R³R⁴⁺, or to general formulae R¹R²N═CR³R⁴⁺ and R¹R²P═CR³R⁴⁺ where
for Q₁⁺: R¹, R², R³ and R⁴, identical or different, represent hydrogen, with the exception of the cation NH₄⁺ for NR¹R²R³R⁴⁺, and preferably a single substituent can represent the hydrogen atom, or hydrocarbyl radicals having 1 to 12 carbon atoms, for example alkyl groups, saturated or unsaturated, cycloalkyls or aromatics, aryl or aralkyl, comprising from 1 to 12 carbon atoms,
and for Q₂⁺ at least one substituent R¹, R², R³ or R⁴ contains at least one alcohol function being defined as an OH group grafted onto the cation Q₂⁺, either directly onto a carbon of the cation Q₂⁺, or via an aryl, aralkyl, alkyl or cycloalkyl group, preferably comprising from 1 to 12 carbon atoms, the substituents not having an alcohol function, identical or different, are defined as previously for Q₁⁺,

The quaternary ammoniums and/or phosphoniums of general formula Q₁⁺ and Q₂⁺ can also be derived from nitrogen-containing (imidazolium, pyridinium, pyrrolidinium, pyrazolium, triazolium) or phosphorus-containing heterocycles comprising 1, 2 or 3 nitrogen and/or phosphorus atoms, of general formulae:

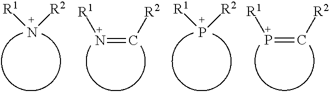

in which the rings are constituted by 4 to 10 atoms, preferably 5 to 6 atoms, R¹ and R² for Q₁⁺ or Q₂⁺ being defined as previously.

The quaternary ammoniums and/or phosphoniums of general formula Q₁⁺ and Q₂⁺ can also consist of a cation corresponding to one of general formulae:

R¹R²⁺N═CR³—R⁵—R³C═N⁺R¹R² and

R¹R²⁺P=CR³—R⁵—R³C═P⁺R¹R²

in which R¹, R² and R³, identical or different, are defined as previously for Q₁⁺ or Q₂⁺ and R⁵ represents an alkylene or phenylene radical.

Among the R¹, R², R³ and R⁴ groups the methyl, ethyl, propyl, isopropyl, butyl, sec-butyl, tert-butyl, amyl, phenyl or benzyl radicals will be mentioned; R⁵ can be a methylene, ethylene, propylene or phenylene group.

Among the R¹, R², R³ and R⁴ groups containing an alcohol function, the —CH₂OH, —(CH₂)₂OH, —(CH₂)₃OH, —(CH₂)₄OH, —C(CH₃)₂—CH₂OH, —CH₂—C(CH₃)₂OH, —C(CH₃)H—CH₂OH, —C₆H₄—CH₂OH radicals will be mentioned.

The quaternary ammonium and/or phosphonium cation Q₁⁺ is preferably chosen form the group formed by N-butylpyridinium, N-ethylpyridinium, 1-butyl-3-methylimidazolium, diethylpyrazolium, 1-ethyl-3-methylimidazolium, pyridinium, trimethylphenylammonium, tetrabutylphosphonium, N-ethyl-N-methylpyrrolidinium and N-butyl-N-ethylpyrrolidinium.

The quaternary ammonium and/or phosphonium cation Q₂⁺ is preferably chosen from the group formed by 1-butyl-3-(2-hydroxyethyl)imidazolium, 1-ethyl-3-(2-hydroxyethyl)imidazolium, N-butyl-N-(2-hydroxyethyl)pyrrolidinium, N-ethyl-N-(2-hydroxyethyl)pyrrolidinium, (2-hydroxyethyl)triethylammonium and triphenyl(3-hydroxypropyl)phosphonium.

The trialkylsulphoniums Q₁⁺ or Q₂⁺ considered in the invention have a general formula of SR¹R²R³⁺ in which
for Q₁⁺: R¹, R² and R³ identical or different, represent hydrocarbyl radicals having 1 to 12 carbon atoms, for example alkyl or alkenyl, cycloalkyl or aromatic, aryl or aralkyl groups, comprising from 1 to 12 carbon atoms,
and for Q₂⁺: at least one substituent R¹, R² or R³ represents an alcohol function being defined as an OH group grafted onto the cation Q₂⁺, either directly onto a carbon of the cation Q₂⁺, or via an aryl, aralkyl, alkyl or cycloalkyl group, preferably comprising from 1 to 12 carbon atoms, the substituents not having an alcohol function, identical or different, are defined as previously for Q₁⁺.

As examples of the ionic liquids Q₁⁺A₁⁻ which can be used, N-butylpyridinium hexafluorophosphate, N-ethylpyridinium tetrafluoroborate, 1-butyl-3-methylimidazolium hexafluoroantimonate, 1-butyl-3-methylimidazolium hexafluorophosphate, 1-butyl-3-methylimidazolium trifluoromethylsulphonate, pyridinium fluorosulphonate, trimethylphenylammonium hexafluorophosphate, 1-butyl-3-methylimidazolium bis-trifluoromethylsulphonylamide, N-ethyl-N-methylpyrrolidinium bis-trifluoromethylsulphonylamide, triethylsulphonium bis-trifluoromethylsulphonylamide, tributylhexylammonium bis-trifluoromethylsulphonylamide, 1-butyl-3-methylimidazolium trifluoroacetate and 1-butyl-2,3-dimethylimidazolium bis-trifluoromethylsulphonylamide can be mentioned.

These ionic liquids can be used alone or in a mixture. They have a solvent function.

As examples of the additives Q₂⁺A₂⁻ which can be used, N-(4-hydroxybutyl)pyridinium hexafluorophosphate, N-(2-hydroxyethyl)pyridinium tetrafluoroborate, 1-(4-hydroxybutyl)-3-methylimidazolium hexafluoroantimonate, 1-(4-hydroxybutyl)-3-methylimidazolium hexafluorophosphate, 1-(4-hydroxybutyl)-3-methylimidazolium trifluoromethylsulphonate, N-(2-hydroxyethyl)pyridinium fluorosulphonate, trimethyl(2-hydroxyethyl)ammonium hexafluorophosphate, 1-(4-hydroxybutyl)-3-methylimidazolium bis-trifluoromethylsulphonylamide, N-(2-hydroxyethyl)-N-methylpyrrolidinium bis-trifluoromethylsulphonylamide, diethyl(2-hydroxyethyl)sulphonium bis-trifluoromethylsulphonylamide, tri butyl (4-hydroxybutypammonium bis-trifluoromethylsulphonylamide, 1-(4-hydroxybutyl)-3-methylimidazolium trifluoroacetate, 1-(4-hydroxybutyl)-2,3-dimethylimidazolium bis-trifluoromethylsulphonylamide and 1-butyl-3-(2-hydroxyethyl)imidazolium bis-trifluoromethylsulphonylamide can be mentioned.

These additives can be used alone or in a mixture. They make it possible to improve the dimer selectivity and are recyclable.

Preferably, the anions A₁⁻ and A₂⁻ are identical in the catalytic composition used according to the invention. The molar ratio between the additive Q₂⁺A₂⁻ and the ionic liquid Q₁⁺A₁⁻ is preferably less than 2/1, and yet more preferably less than 1/1.

The Brønsted acids are defined as being organic acid compounds capable of donating at least one proton. These Brønsted acids have a general formula HB, in which B represents an anion.

The anions B are preferably chosen from tetrafluoroborate, tetraalkylborates, hexafluorophosphate, hexafluoroantimonate, alkylsulphonates and arylsulphonates (for example methylsulphonate or tosylate), perfluoroalkylsulphonates (for example trifluoromethylsulphonate), fluorosulphonate, sulphates, phosphates, perfluoroacetates (for example trifluoroacetate), perfluoroalkylsulphonamides (for example bis-trifluoromethanesulphonyl amide (N(CF₃SO₂)₂⁻), fluorosulphonamides, perfluoroalkylsulphomethides (for example tris-trifluoromethanesulphonyl methylide (C(CF₃SO₂)₃⁻) and carboranes.

The Brønsted acids can be used alone or in a mixture. Preferably, the anions B, A₁⁻ and A₂⁻ are identical in the formula of the Brønsted acids used.

In all cases, the molar ratio of the Brønsted acid to the sum of (Q₁⁺A₁⁻+Q₂⁺A₂⁻) is less than 2/1, preferably less than 1/1.

The compounds involved in the catalytic composition used in the process of the invention can be mixed in any order. The mixing can be done by simply bringing them into contact followed by stirring until a homogeneous liquid is formed. This mixing can be done outside the reactor used for the catalytic application or inside this reactor.

Also a subject of the present invention is an isobutene dimerization process using the catalytic composition. The dimerization process according to the invention applies to pure isobutene or isobutene in a mixture with other hydrocarbons.

The sources of isobutene are varied. However, the most common are the dehydrogenation of isobutane and the dehydration of tert-butyl alcohol The isobutene can also originate from a C4 cut from catalytic cracking in a fluidized bed or from steam cracking. In this latter case, the isobutene can be used in a mixture with n-butenes, isobutane and butane. The process according to the present invention then has the additional advantage of making it possible to selectively convert the isobutene without having to separate the other constituents of the cut. Another advantage of the process according to the invention is that the isobutene-butene co-dimerization can be limited.

Recent advances in the field of biotechnologies show that it is also possible to produce isobutene from 2-methyl-propanol (isobutanol), itself obtained from sugars originating from the fermentation of biomass.

The ratio by volume of the isobutene to the catalytic composition can be comprised between 0.1/1 and 1000/1, preferably between 1/1 and 100/1. It is chosen so as to obtain the best selectivities.

The reaction can be carried out in a closed system, in a semi-open system or continuously with one or more reaction stages. At the reactor outlet, the organic phase containing the reaction products is separated.

In the dimerization process of the invention, an organic solvent such as an aliphatic hydrocarbon or an aromatic hydrocarbon immiscible or partially miscible with the ionic liquid can be added to the catalytic composition which allows a better separation of the phases. As aliphatic hydrocarbon for example pentane, heptane, cyclohexane, decane, dodecane or a paraffinic feedstock can be used, alone or in a mixture. As aromatic hydrocarbon for example toluene or xylene can be used, alone or in a mixture.

The temperature at which the dimerization reaction is carried out ranges for example from −50° C. to 200° C.; it is advantageously below 100° C.

The reaction can be carried out at autogeneous pressure, this can also be increased up to 10 MPa.

The dimerization reaction can be carried out using a reactive distillation technique.

The products obtained by the present invention can be subsequently converted according to different reactions, such as hydrogenation, hydroformylation, oxidation, etherification, epoxidation or hydration.

The following examples illustrate the invention without limiting its scope.

### EXAMPLE 1 (ACCORDING TO THE INVENTION)

### Preparation of the additive 1-butyl-3-(2-hydroxyethyl)imidazolium bis(trifluoromethylsulphonyl)amide [BuIm(CH₂)₂OH][N(CF₃SO₂)₂] designated “(Q₂⁺A₂⁻)” according to the invention

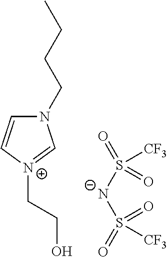

20 g (0.16 mol) of N-butylimidazole as well as 25.9 g (0.32 mol) of 1-chloroethanol are introduced under an inert atmosphere into a 500 ml flask. Then 150 mL of acetonitrile is added. The mixture is refluxed for 5 days. After returning to ambient temperature, the acetonitrile is removed under vacuum. A viscous yellow-coloured liquid is obtained. This compound is dissolved in 100 mL of dichloromethane. Then 48.2 g (0.168 mol) of lithium bis(trifluoromethylsulphonyl)amide is added. After reaction for 2 h at ambient temperature, the mixture is filtered on “neutral celite 545/Al₂O₃” before the solvent is evaporated off under dynamic vacuum. The liquid residue is washed several times with water then dried at 80° C. under dynamic vacuum. 34.5 g (yield 48%) of [BuIm(CH₂)₂OH][N(CF₃SO₂)₂] is obtained. ¹H and ¹³C NMR analyses confirm the structure of the expected product.

### EXAMPLE 2 (ACCORDING TO THE INVENTION)

### Preparation of the “[BuIm(CH₂)₂OH][N(CF₃SO₂)₂]/[BMIm][N(CF₃SO₂)₂]/HN(CF₃SO₂)₂” catalytic system designated “Q₂⁺A₂⁻/Q₁⁺A₁⁻/HB” according to the invention

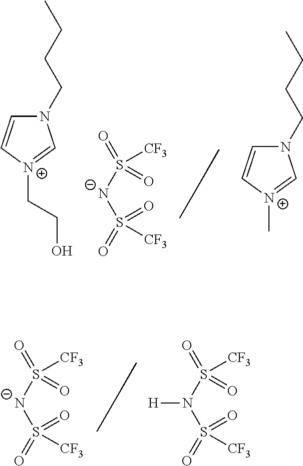

4.97 g (11.9 mmol) of 1-butyl-3-methylimidazolium bis(trifluoromethylsulphonyl)amide [BMIm][N(CF₃SO₂)₂)] is mixed at ambient temperature, under an inert atmosphere, with 0.85 g (1.9 mmol) of [BuIm(CH₂)₂OH][N(CF₃SO₂)₂] described in Example 1. The mixture represents a volume of 4 mL. Then 0.014 g (0.051 mmoles) of bis-triflylamide acid HN(CF₃SO₂)₂ is added. The mixture is stirred for a few minutes and leads to a clear solution containing 0.013 mol/L of acid.

### EXAMPLE 3 (COUNTER EXAMPLE)

### Preparation of the [BMIm][N(CF₃SO₂)₂]/HN(CF₃SO₂)₂ catalytic system designated “Q₁⁺A₁⁻/HB”

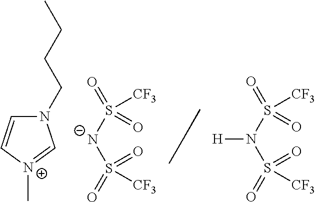

4 mL (5.5 g, 13.1 mmol) of 1-butyl-3-methylimidazolium bis(trifluoromethylsulphonyl)amide [BMIm][N(CF₃SO₂)₂) is mixed at ambient temperature, under an inert atmosphere, with 0.015 g (0.054 mmoles) of bis-triflylamide acid HN(CF₃SO₂)₂. The mixture is stirred for a few minutes and leads to a clear solution containing 0.013 mol/L of acid.

### EXAMPLE 4 (COUNTER EXAMPLE)

### Preparation of the [BuIm(CH₂)₂OH][N(CF₃SO₂)₂]/HN(CF₃SO₂)₂ catalytic system designated “Q₂⁺A₂⁻/HB”

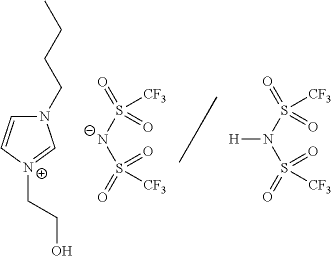

4 mL (4.8 g, 10.7 mmol) of [BuIm(CH₂)₂OH][N(CF₃SO₂)₂] described in Example 1 is mixed at ambient temperature, under an inert atmosphere, with 0.014 g (0.013 mmoles) of bis-triflylamide acid HN(CF₃SO₂)₂. The mixture is stirred for a few minutes and leads to a clear solution containing 0.013 mol/L of acid.

### EXAMPLE 5 (ACCORDING TO THE INVENTION)

### Dimerization of Isobutene Using the Catalytic Composition of Example 2

All of the mixture prepared in Example 2 is introduced, under an argon atmosphere, into a 100 mL Fisher-Porter tube, provided with a magnetic stirring bar and dried and brought under vacuum before the study. Then, 20 mL (11.2 g) of a liquid feedstock containing 95% isobutene and 5% n-butane is introduced, at ambient temperature. Stirring is then started (reaction time zero). After reaction for 20 minutes at 25° C., the stirring is stopped. The supernatant organic phase is separated from the ionic liquid phase and analyzed by GC (gas chromatography, with heptane as external standard) after treatment with soda (10N) in order to remove any traces of acid and drying over MgSO₄. The isobutene conversion is 73%. The dimerization product selectivity (C8) is 74%, the trimer selectivity (C12) is 23% and the tetramer selectivity (C16) is 3%.

The ionic liquid phase containing the [BuIm(CH₂)₂OH][N(CF₃SO₂)₂]/[BMIm][N(CF₃SO₂)₂]/HN(CF₃SO₂)₂ mixture was isolated and reused over several catalysis cycles without adjustment of the catalytic composition (Examples 6 to 11). The results obtained are shown in Table 1.

### EXAMPLE 12 (COUNTER EXAMPLE)

### Dimerization of Isobutene Using the Catalytic Composition of Example 3

The protocol is identical to that described in Example 5 except that the catalytic composition described in Example 3 is used. After reaction for 7 minutes at 25° C., the stirring is stopped. The supernatant organic phase is separated from the ionic liquid phase and analyzed by GC. The isobutene conversion is 84%. The dimerization product selectivity (C8) is 47%, the trimer selectivity (C12) is 46% and the tetramer selectivity (C16) is 6% (see Table 2).

### EXAMPLE 13 (COUNTER EXAMPLE)

### Dimerization of Isobutene Using the Catalytic Composition of Example 4

The protocol is identical to that described in Example 5 except that the catalytic composition described in Example 4 is used. After reaction for 20 minutes at 25° C., the stirring is stopped. The supernatant organic phase is separated from the ionic liquid phase and analyzed by GC. No conversion of isobutene was observed (see Table 2).

TABLE 1

| | | | | Conv. | Selectivities (wt. %) | | | |
|---|---|---|---|---|---|---|---|---|
| Ex | Q₁⁺A₁⁻ | Q₂⁺A₂⁻ | HB | iC₄⁼ | C8 | C12 | C16 | C16+ |
| 5 | [BMIm] | [BuIm(CH₂)₂OH] | HN(CF₃SO₂)₂ | 73 | 74 | 23 | 3 | <0.5 |
| | [N(CF₃SO₂)₂] | [N(CF₃SO₂)₂] | | | | | | |

| | | | | | | |
|---|---|---|---|---|---|---|
| 6 | Recycling | 75 | 71 | 26 | 3 | <0.5 |
| 7 | Recycling | 79 | 71 | 26 | 3 | <0.5 |
| 8 | Recycling | 79 | 71 | 26 | 3 | <0.5 |
| 9 | Recycling | 67 | 74 | 23 | 3 | <0.5 |
| 10 | Recycling | 72 | 73 | 23 | 4 | <0.5 |
| 11 | Recycling | 73 | 74 | 23 | 3 | <0.5 |

TABLE 2

| | | | | Conv. | Selectivities (wt. %) | | | |
|---|---|---|---|---|---|---|---|---|
| Ex | Q₁⁺A₁⁻ | Q₂⁺A₂⁻ | HB | iC₄⁼ | C8 | C12 | C16 | C16+ |
| 12 | [BMIm] | — | HN(CF₃SO₂)₂ | 84 | 47 | 26 | 6 | <0.5 |
| | [N(CF₃SO₂)₂] | | | | | | | |
| 13 | — | [BuIm(CH₂)₂OH] | HN(CF₃SO₂)₂ | — | — | — | — | <0.5 |
| | | [N(CF₃SO₂)₂] | | | | | | |

The catalytic composition containing the additive Q₂⁺A₂⁻ according to the invention makes it possible to obtain a better selectivity of C8 dimers compared to a catalytic composition without additive. It can also be seen that the catalytic system is recyclable.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosures of all applications, patents and publications, cited herein and of corresponding French application Ser. No. 11/03654, filed Nov. 30, 2011, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Catalytic composition comprising at least one Brønsted acid, designated HB, dissolved in a non-aqueous liquid medium of general formula Q₁⁺A₁⁻, in which Q₁⁺ represents an organic cation and A₁⁻ represents an anion, said composition also comprising an additive Q₂⁺A₂⁻ in which Q₂⁺ represents an organic cation comprising at least one alcohol function and A₂⁻ represents an anion.

2. Catalytic composition according to claim 1, in which the anion A₁⁻ or A₂⁻ is chosen from the following anions: tetrafluoroborate, tetraalkylborates, hexafluorophosphate, hexafluoroantimonate, alkylsulphonates, arylsulphonates, perfluoroalkylsulphonates, fluorosulphonate, sulphates, phosphates, perfluoroacetates, perfluoroalkylsulphonamides, fluorosulphonamides, perfluoroalkylsulphomethides and carboranes, A₁⁻ and A₂⁻ being identical or different.

3. Catalytic composition according to claim 1, in which Q₁⁺ represents a quaternary ammonium and/or a quaternary phosphonium and/or a trialkylsulphonium, and Q₂⁺ represents a quaternary ammonium and/or a quaternary phosphonium and/or a trialkylsulphonium comprising at least one alcohol function, and A₁⁻ or A₂⁻ represent any anion known to be non-coordinating and capable of forming a liquid salt below 150° C.

4. Catalytic composition according to claim 3, in which the ammonium and/or quaternary phosphonium cation Q₁⁺ or Q₂⁺ is chosen from:
the ammonium and/or quaternary phosphonium cations corresponding to one of the general formulae NR¹R²R³R⁴⁺ and PR¹R²R³R⁴⁺, or to one of the general formulae R¹R²N═CR³R⁴⁺ and R¹R²P═CR³R⁴⁺ where,
for Q₁⁺: R¹, R², R³ and R⁴, identical or different, represent hydrogen, with the exception of the cation NH₄⁺ for NR¹R²R³R⁴⁺, a single substituent representing the hydrogen atom, or hydrocarbyl radicals having 1 to 12 carbon atoms,
and for Q₂⁺: at least one substituent R¹, R², R³ or R⁴ contains at least one alcohol function being defined as an OH group grafted onto the cation Q₂⁺, either directly onto a carbon of the cation Q₂⁺, or via an aryl, aralkyl, alkyl or cycloalkyl group, preferably comprising from 1 to 12 carbon atoms, the substituents not having an alcohol function, identical or different, are defined as previously for Q₁⁺,
the quaternary ammonium and/or phosphonium cations derived from nitrogen-containing or phosphorus-containing heterocycles comprising 1, 2 or 3 nitrogen and/or phosphorus atoms, of general formulae: 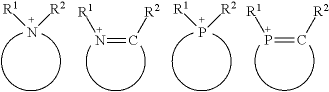
in which the rings are constituted by 4 to 10 atoms, preferably 5 to 6 atoms, R¹ and R² for Q₁⁺ or Q₂⁺ being defined as previously,
the quaternary ammonium and/or phosphonium cations corresponding to one of general formulae:
R¹R²⁺N═CR³—R⁵—R³C═N⁺R¹R²
R¹R²⁺P═CR³—R⁵—R³C═P⁺R¹R²
in which R¹, R² and R³, identical or different, are defined as previously for Q₁⁺ or Q₂⁺ and R⁵ represents an alkylene or phenylene radical.

5. Catalytic composition according to claim 4, in which the quaternary ammonium and/or phosphonium cation Q₁⁺ is chosen from the group formed by N-butylpyridinium, N-ethylpyridinium, 1-butyl-3-methylimidazolium, diethylpyrazolium, 1-ethyl-3-methylimidazolium, pyridinium, trimethylphenylammonium, tetrabutylphosphonium, N-ethyl-N-methylpyrrolidinium and N-butyl-N-ethylpyrrolidinium.

6. Catalytic composition according to claim 4, in which the quaternary ammonium and/or phosphonium cation Q₂⁺ is chosen from the group formed by 1-butyl-3-(2-hydroxyethyl)imidazolium, 1-ethyl-3-(2-hydroxyethyl)imidazolium, N-butyl-N-(2-hydroxyethyl)pyrrolidinium, N-ethyl-N-(2-hydroxyethyl)pyrrolidinium, (2-hydroxyethyl)triethylammonium and triphenyl(3-hydroxypropyl)phosphonium.

7. Catalytic composition according to claim 3, in which Q₁⁺ or Q₂⁺ is a trialkylsulphonium cation corresponding to general formula SR¹R²R³⁺ in which
for Q₁⁺: R¹, R² and R³, identical or different, represent hydrocarbyl radicals having 1 to 12 carbon atoms,
and for Q₂⁺: at least one substituent R¹, R² or R³ represents an alcohol function being defined as an OH group grafted onto the cation Q₂⁺, either directly onto a carbon of the cation Q₂⁺, or via an aryl, aralkyl, alkyl or cycloalkyl group, preferably comprising form 1 to 12 carbon atoms, the substituents not having an alcohol function, identical or different, are defined as previously for Q₁⁺.

8. Catalytic composition according to claim 1, in which the ionic liquid Q₁⁺A₁⁻ is chosen from N-butylpyridinium hexafluorophosphate, N-ethyl pyridinium tetrafluoroborate, 1-butyl-3-methylimidazolium hexafluoroantimonate, 1-butyl-3-methylimidazolium hexafluorophosphate, 1-butyl-3-methylimidazolium trifluoromethylsulphonate, pyridinium fluorosulphonate, trimethylphenylammonium hexafluorophosphate, 1-butyl-3-methylimidazolium bis-trifluoromethylsulphonylamide, N-ethyl-N-methylpyrrolidinium bis-trifluoromethylsulphonylamide, triethylsulphonium bis-trifluoromethylsulphonylamide, tributylhexylammonium bis-trifluoromethylsulphonylamide, 1-butyl-3-methylimidazolium trifluoroacetate and 1-butyl-2,3-dimethylimidazolium bis-trifluoromethylsulphonylamide, alone or in a mixture.

9. Catalytic composition according to claim 1, in which the additive Q₂⁺A₂⁻ is chosen from N-(4-hydroxybutyl)pyridinium hexafluorophosphate, N-(2-hydroxyethyl)pyridinium tetrafluoroborate, 1-(4-hydroxybutyl)-3-methylimidazolium hexafluoroantimonate, 1-(4-hydroxybutyl)-3-methylimidazolium hexafluorophosphate, 1-(4-hydroxybutyl)-3-methylimidazolium trifluoromethylsulphonate, N-(2-hydroxyethyl)pyridinium fluorosulphonate, trimethyl (2-hydroxyethyl) ammonium hexafluorophosphate, 1-(4-hydroxybutyl)-3-methylimidazolium bis-trifluoromethylsulphonylamide, N-(2-hydroxyethyl)-N-methylpyrrolidinium bis-trifluoromethylsulphonylamide, diethyl(2-hydroxyethyl)sulphonium bis-trifluoromethylsulphonylamide, tributyl(4-hydroxybutyl)ammonium bis-trifluoromethylsulphonylamide, 1-(4-hydroxybutyl)-3-methylimidazolium trifluoroacetate, 1-(4-hydroxybutyl)-2,3-dimethylimidazolium bis-trifluoromethylsulphonylamide and 1-butyl-3-(2-hydroxyethyl)imidazolium bis-trifluoromethylsulphonylamide, alone or in a mixture.

10. Catalytic composition according to claim 1, in which the molar ratio between the additive Q₂⁺A₂⁻ and the ionic liquid Q₁⁺A₁⁻ is less than 2/1.

11. Catalytic composition according to claim 1, in which the anion B of the Brønsted acid is chosen from the following anions: tetrafluoroborate, tetraalkylborates, hexafluorophosphate, hexafluoroantimonate, alkylsulphonates, perfluoroalkylsulphonates, fluorosulphonate, sulphates, phosphates, perfluoroacetates, perfluoroalkylsulphonamides, fluoro sulphonamides, perfluoroalkylsulphomethides and carboranes.

12. Catalytic composition according to claim 1, in which the molar ratio of the Brønsted acid to the sum of (Q₁⁺A₁⁻+Q₂⁺A₂⁻) is less than 2/1.

13. Isobutene dimerization process using a catalytic composition according to claim 1.

14. Isobutene dimerization process according to claim 13 in which the ratio by volume of the isobutene to the catalytic composition is comprised between 0.1/1 and 1000/1.

15. Isobutene dimerization process according to claim 13 in which the dimerization reaction is carried out at a temperature between −50° C. to 200° C., at a pressure ranging from autogeneous pressure to 10 MPa.
